(19) United States
(12) Patent Application Publication
(10) Pub. No.: US 2020/0181562 A1
(43) Pub. Date: Jun. 11, 2020

(54) CELL ISOLATION FOR USE IN AUTOMATED BIOREACTORS

(71) Applicant: LONZA WALKERSVILLE, INC., Walkersville, MD (US)

(72) Inventors: Erika MCAFEE, Pearland, TX (US); Yaling SHI, Gaithersburg, MD (US); Samatha BANDAPALLE, Monrovia, MD (US); Eytan ABRAHAM, Potomac, MD (US)

(21) Appl. No.: 16/710,952

(22) Filed: Dec. 11, 2019

Related U.S. Application Data
(60) Provisional application No. 62/778,078, filed on Dec. 11, 2018.

(51) Int. Cl. C12M 1/00; C12M 1/36; C12M 3/00

(52) U.S. Cl. CPC C12M 47/04; C12M 23/44; C12M 41/48; C12M 21/00

(57) ABSTRACT

The present disclosure provides cassettes for use in automated cell engineering systems that include cell separation filters for capturing a target cell population for automated processing. The disclosure also provides methods of separating a target cell population, as well as automated cell engineering systems using the cassettes and for carrying out the methods.

### FIELD OF THE INVENTION

The present disclosure provides cassettes for use in automated cell engineering systems that include cell separation filters for capturing a target cell population for automated processing. The disclosure also provides methods of separating a target cell population, as well as automated cell engineering systems that can utilize the cassettes and carry out the methods.

### BACKGROUND OF THE INVENTION

As anticipation builds about accelerated clinical adoption of advanced cell therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields.

Automation of various processes is involved in producing cell populations for therapy. This includes integration of cell activation, transduction and expansion into a commercial manufacturing platform for the translation of these important therapies to the broad patient population.

In addition, it is highly desired in automated cell processing platforms to limit the number of times, or steps, in which a cell population is exposed to the external environment so as to limit contamination and other problems. What is needed is a process by which a cellular sample can be directly provided to an automated system, where any cellular isolation or cell filtration is carried out within the automated system, and thus the total number of steps when cells are exposed to the environment can be potentially limited to just introduction and collection following the various automated processes.

### SUMMARY OF THE INVENTION

In some embodiments provided herein is a cassette for use in an automated cell engineering system, comprising a cellular sample input, a cell separation filter fluidly connected to the cellular sample input, a cell culture chamber fluidly connected to the cell separation filter, and a cellular sample output fluidly connected to the cell culture chamber. Suitably, the cassette does not include a centrifuge following the cell separation filter.

In additional embodiments, a cassette for use in an automated cell engineering system, comprising a cellular sample input, a cell separation filter fluidly connected to the cellular sample input, the cell separation filter including a matrix which captures immune cells, a cell culture chamber for carrying out activation, transduction and/or expansion of the immune cells having a chamber volume that is configured to house the immune cells, a back flush system fluidly connected to the cell separation filter, and a cellular sample output fluidly connected to the cell culture chamber. Suitably, the cassette does not include a centrifuge following the cell separation filter.

In additional embodiments, provided herein is a method of preparing a target cell population for automated processing, the method comprising introducing a cellular sample containing the target cell population into a cassette of an automated cell engineering system, passing the cellular sample through a cell separation filter, capturing the target cell population from the cellular sample onto a matrix of the cell separation filter, back flushing the cell separation filter, and transferring the target cell population from the cell separation filter, so that the target cell population can undergo automated processing.

Also provided herein is an automated cell engineering system, comprising an enclosable housing, a cassette contained within the enclosable housing, the cassette comprising a cellular sample input, a cell separation filter fluidly connected to the cellular sample input, a cell culture chamber fluidly connected to the cell separation filter, and a cellular sample output fluidly connected to the cell culture chamber, wherein the cassette does not include a centrifuge following the cell separation filter, and a user interface for receiving input from a user.

### BRIEF DESCRIPTION OF THE FIGURES

FIG. 1 shows various steps that can be performed with a cassette of an automated cell engineering system, as described in embodiments hereof.

FIG. 2A shows an exemplary cassette in accordance with embodiments hereof.

FIGS. 2B and 2C show exemplary cell separation filters in accordance with embodiments hereof.

FIGS. 3A and 3B show images of an automated cell engineering system in accordance with embodiments hereof.

FIG. 4 shows a lab space containing exemplary cell engineering systems as described in embodiments herein.

FIG. 5 shows a flowpath for cell separation and isolation in a cassette of an automated cell engineering system as described in embodiments herein.

FIG. 6A shows comparison of Donor 1 cell viability (%) post leukocyte isolation via whole blood cell isolation Ficoll and cell separation filtration methods.

FIG. 6B shows comparison of Donor 1 total cell yield post whole blood Ficoll and cell separation filtration processing.

FIG. 7A shows total cell yield over 11 days of culture post whole blood processing via Ficoll and filtration methods.

FIG. 7B shows average culture viability (%) of duplicate T-25 flask culture post whole blood processing via Ficoll and filtration.

FIG. 8A shows comparison of Donor 2 cell viability (%) post leukocyte isolation via whole blood cell isolation Ficoll and filtration methods.

FIG. 8B shows comparison of Donor 2 total cell yield post whole blood Ficoll and filtration processing.

FIG. 9A shows comparison of Leukopak Donor cell viability (%) post leukocyte isolation via Ficoll and filtration methods.

FIG. 9B shows comparison of Leukopak Donor total cell yield post whole blood Ficoll and filtration processing.

FIG. 10 shows gating strategy for FACS analysis.

FIG. 11 shows percentage of CD3+CD4+ and CD3+CD8+ T-cells from Donor 1 filtered and Ficoll isolated whole blood collection samples.

FIG. 12 shows percentage of CD3+CD4+ and CD3+CD8+ T-cells from Donor 2 filtered and Ficoll isolated whole blood collection samples.

FIG. 13 shows percentage of CD3+CD4+ and CD3+CD8+ T-cells from filtered and Ficoll isolated leukopak collection samples.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

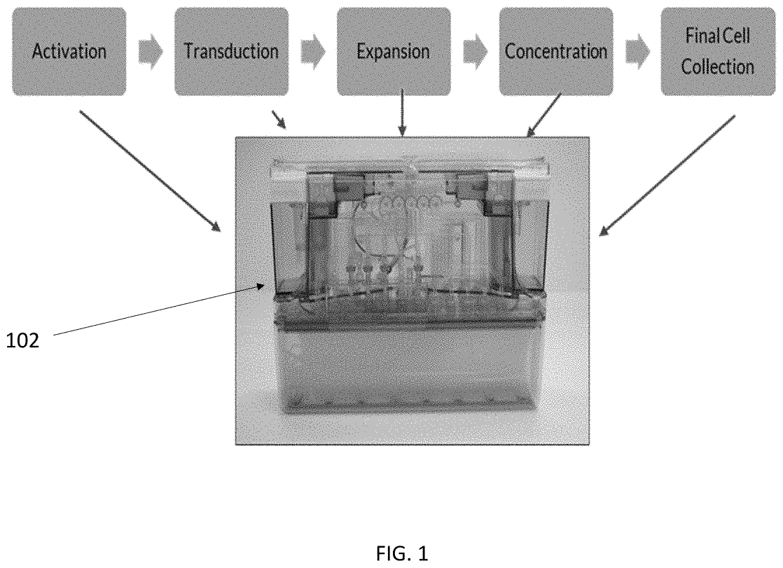

In embodiments, provided herein are cassettes for use in an automated cell engineering system. FIG. 1 shows an exemplary cassette 102, in which various processes can be carried out in an enclosed, automated system that allows for production of various cellular samples and populations. Such processes can include activating, transducing, expanding, concentrating, and collecting/harvesting steps

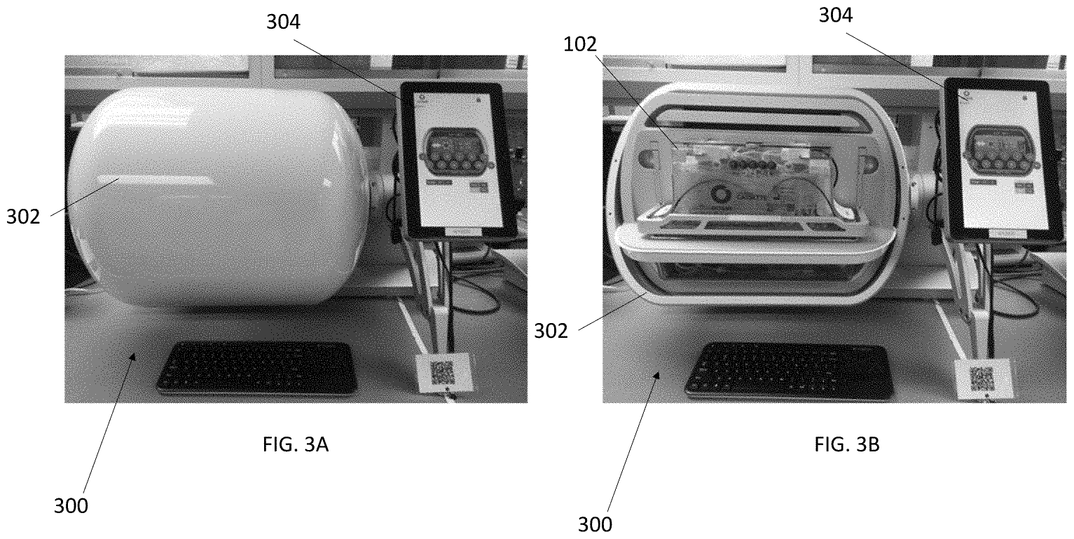

As described herein, the cassettes and methods are suitably utilized and carried out in a fully enclosed automated cell engineering system 300 (see FIGS. 3A, 3B), suitably having instructions thereon for performing steps such as, activating, transducing, expanding, concentrating, and harvesting. Cell engineering systems for automated production of, for example genetically modified immune cells, including CAR T cells, are described in U.S. patent application Ser. No. 16/119,618, filed Aug. 31, 2018 (the disclosure of which is incorporated by reference herein in its entirety), and are also called automated cell engineering system, COCOON, or COCOON system herein.

For example, a user can provide a automated cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like), the automated cell engineering system is able to carry out the various automated methods, including methods of producing genetically modified immune cell cultures, including CAR T cells, without further input from the user. In some embodiments, the fully enclosed automated cell engineering system minimizes contamination of the cell cultures by reducing exposure of the cell culture to non-sterile environments. In additional embodiments, the fully enclosed automated cell engineering system minimizes contamination of the cell cultures by reducing user handling of the cells.

As described herein, the automated cell engineering systems 300 suitably include a cassette 102. Thus, in embodiments, provided herein is a cassette for use in an automated cell engineering system. As used herein a “cassette” refers to a largely self-contained, removable and replaceable element of a automated cell engineering system that includes one or more chambers for carrying out the various elements of the methods described herein, and suitably also includes one or more of a cell media, an activation reagent, a wash media, etc.

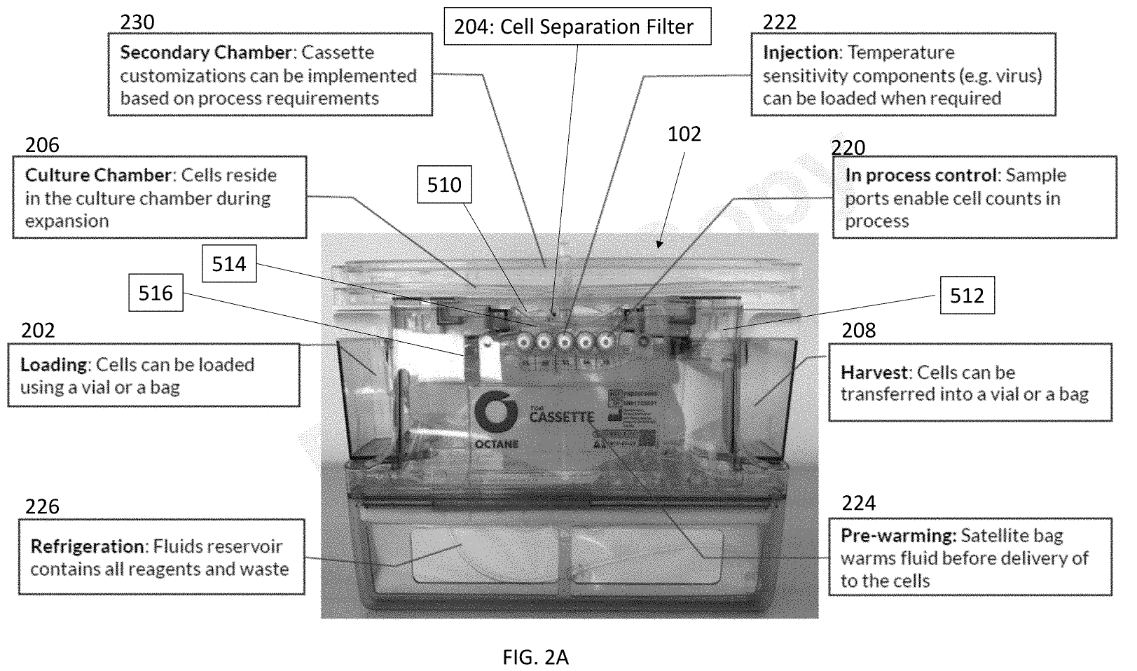

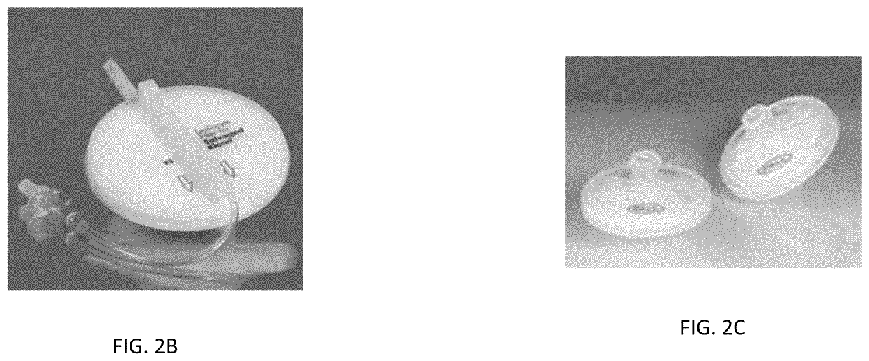

FIG. 2A shows an exemplary cassette 102 for use in an automated cell engineering system. In embodiments, cassette 102 includes a cellular sample input 202. Cellular sample input 202 is shown in FIG. 2A as a vial or chamber in which a cellular sample can be placed prior to introduction or loading into cassette 102. In other embodiments, cellular sample input 202 can simply be a sterile-locking tubing (for example a luer lock tubing connection or the like) to which a syringe or a cell-containing bag, such as a blood bag, can be connected.

Cassette 102 further includes a cell separation filter 204, located within the cassette, and fluidly connected to cellular sample input 202. As used herein, “fluidly connected” means that one or more components of a system, including cassette 102, are connected via a suitable element that allows for fluids (including gasses and liquids) to pass between the components without leaking or losing volume. Exemplary fluid connections include various tubing, channels and connections known in the art, such as silicone or rubber tubing, luer lock connections, etc. It should be understood that components that are fluidly connected can also include additional elements between each of the components, while still maintaining a fluid connection. That is, fluidly connected components can include additional elements, such that a fluid passing between the components can also pass through these additional elements, but is not required to do so.

Cassette 102 suitably further includes a cell culture chamber 206 fluidly connected to the cell separation filter. Examples of the characteristics and uses of cell culture chamber 206 are described herein.

In embodiments, cassette 102 further includes one or more fluidics pathways connected to the cell culture chamber (see inside cassette 102 in FIG. 2A). Also included in cassette 102 is a cellular sample output 208 fluidly connected to cell culture chamber. As described herein, cellular sample output 208 is utilized to harvest the cells following the various automated procedures for either further processing, storage, or potential use in a patient. Examples of fluidics pathways include various tubing, channels, capillaries, microfluidics elements, etc., that provide nutrients, solutions, etc., to the elements of the cassette, as described herein.

As described herein, cassette 102 explicitly excludes a centrifuge following cell separation filter 204. “Following the cell separation filter” includes embodiments where a centrifuge is not included downstream of the cell separation filter, or downstream of the back flush from the cell separation filter. It has been determined that through the use of the various cell separation filters and methods described herein, additional cellular separation via centrifugation procedures and the use of a centrifuge is not required. In embodiments, however, a further filtration system, such as a column filtration, tangential flow filtration, and/or magnetic filtration system, can be utilized.

In exemplary embodiments, cell separation filter 204 includes a matrix which captures a cell population, suitably target cells. Suitable matrix materials include various porous media that has been treated with a gas plasma. The porous media can be a natural or synthetic fiber or woven material, or a sintered powder material. Exemplary matrix materials include those disclosed in, for example, U.S. Pat. Nos. 4,701,267, 4,936,998, 4,880,548, 4,923,620, 4,925,572, and 5,679,264, the disclosures of each of which are incorporated by reference herein in their entireties. As used herein a “target cell population” or “target cells” refers to a desired sub-set of cells that is to be separated from a larger cell population, including from debris or other contaminants, such that the remaining target cell population is largely free of other cell types. Exemplary target cell populations include immune cells, cancer cells, etc.

Exemplary cell separation filters suitably include a matrix that allows for the capture of immune cells, that is the matrix retains immune cells on or within the matrix. As used herein, “immune cells” includes basophils, eosinophils, neutrophils, leukocytes, etc., and include cells such as mast cells, dendritic cells, naturally killer cells, B cell, T cells, etc. As described herein, the cassettes and cell separation filters are suitably used to separate immune cells from a cellular sample, including a whole blood cell sample or a leukophoresis sample (sample in which white blood cells are separated from whole blood).

FIGS. 2B and 2C show exemplary cell separation filters for use in the cassettes and methods described herein. FIG. 2B shows a leukocyte filter for salvaged blood (Haemonetics, Braintree, Mass.) and FIG. 2C a syringe filter (PALL ARCODISC®, PALL Laboratory, Port Washington, N.Y.).

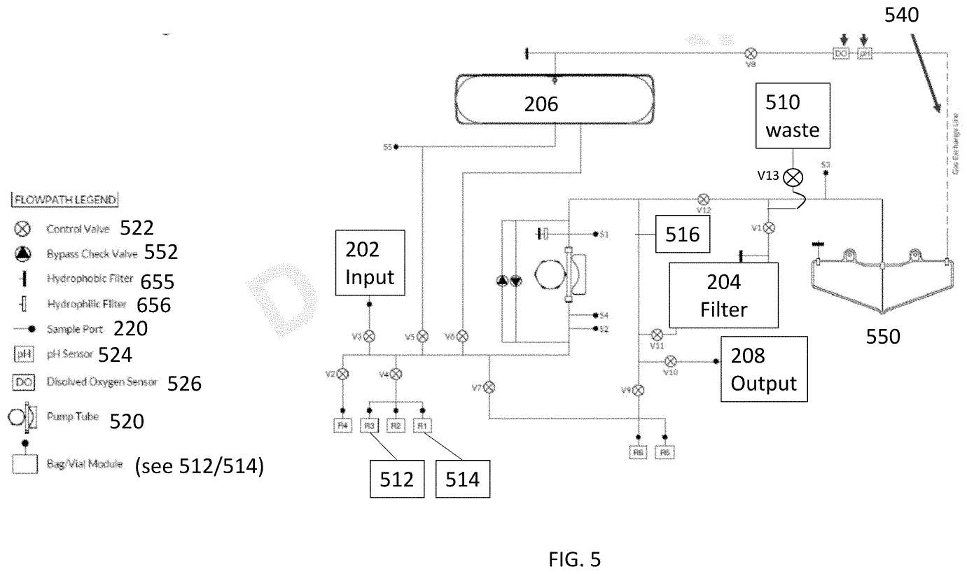

In additional embodiments, cassette 102 suitably includes a waste collection chamber 510 (contained within cassette 102 in FIG. 2A), following cell separation filter 204 and fluidly connected to the separation filter. An exemplary location for waste collection chamber 510 within the flowpath of a cassette is shown in FIG. 5. Waste collection chamber 510 is suitably positioned following, or downstream (i.e., fluidly connected after the cell separation filter) so that waste that passes through cell separation filter can be held for either further processing or disposal. Waste the can be collected suitably includes undesired cells, either whole or lysed, as well as blood components, as well as potential contaminants within a cellular sample that is being filtered. Waste chamber 510 can be in the form of a solid chamber or a bag within cassette 102, or can be a bag or chamber external to the cassette, but connected via a fluidic path, such as tubing and a sampling port.

In embodiments, cassette 102 includes a cell wash system 512 that is suitably contained within cassette 102 (i.e., within the structure shown in FIG. 2A), and fluidly connected to separation filter 204. As shown in FIG. 5, cell wash system 512 can be connected to one of the various input ports of cassette 102, to allow for a direct fluid path to separation filter 204. In embodiments, cell wash system 512 is a container or bag contained within cassette that suitably includes a cell wash media. The cell wash media is suitably used to clean the target cell population and the separation filter and remove any undesired waste cells or contamination from the target cell population, prior to transferring the target cell population from the cell separation filter to another portion of the cassette. Cell wash system 512 can also be included outside of cassette 102. In further embodiments, cell wash system 512 can be used to wash cells held in a target cell population holding chamber.

In additional embodiments cassette 102 includes a back flush system 514 (not visible in FIG. 2 as it is suitably located inside cassette 102), but shown in FIG. 5 as an element of the flowpath for the cassette. Like cell wash system 512, back flush system 514 is suitably a container or bag contained within the cassette and can be connected to one or of the various input ports of cassette 102, to allow a direct fluid path to separation filter 204. Back flush system 514 can also be included external to the cassette. Back flush system 514 is suitably fluidly connected to separation filter 204 in such a way that a back flush media contained within the back flush system can be introduced into or onto cell separation filter 204 in a reverse manner to transfer cells captured by the separation filter from the filter to another section of the cassette, including a holding chamber or a cell culture chamber, as described herein.

Cassette 102 can also further optionally include a target cell population holding chamber 516 (not visible in FIG. 2 as it is located inside cassette 102) located between the cell separation filter and the cell culture chamber. FIG. 5 shows an exemplary location of target cell population holding chamber 516 in the flowpath for the cassette. Target cell population holding chamber 516 is suitably a reservoir or suitable chamber located within the cassette into which a target cell population that has been captured on the separation filter 204, and then back flushed via back flush system 514 to transfer the captured cells to target cell population holding chamber 516.

As described herein, the fluidics pathways, which can include various tubing elements, suitably provide recirculation, removal of waste and homogenous gas exchange and distribution of nutrients to various parts of the cassette, including the cell culture chamber without disturbing cells within the cell culture chamber. Cassette 102 also further includes one or more pumps 520 and related tubing, including peristaltic pumps, for driving fluid through the cassette, as described herein, as well as one or more valves 522, for controlling the flow through the various fluidic pathways (see FIG. 5 for exemplary locations within flowpath).

In exemplary embodiments, as shown in FIG. 2A, cell culture chamber 206 is flat and non-flexible chamber (i.e., made of a substantially non-flexible material such as a plastic) that does not readily bend or flex. The use of a non-flexible chamber allows the cells to be maintained in a substantially undisturbed state. As shown in FIG. 2A, cell culture chamber 206 is oriented so as to allow the immune cell culture to spread across the bottom of the cell culture chamber. As shown in FIG. 2A, cell culture chamber 206 is suitably maintained in a position that is parallel with the floor or table, maintaining the cell culture in an undisturbed state, allowing the cell culture to spread across a large area of the bottom of the cell culture chamber. In embodiments, the overall thickness of cell culture chamber 206 (i.e., the chamber height) is low, on the order of about 0.5 cm to about 5 cm. Suitably, the cell culture chamber has a volume of between about 0.50 ml and about 300 ml, more suitably between about 50 ml and about 200 ml, or the cell culture chamber has a volume of about 180 ml. The use of a low chamber height (less than 5 cm, suitably less than 4 cm, less than 3 cm, or less then 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss. In other embodiments, cell culture chamber 206 is a bag or hard chamber.

As described herein, in exemplary embodiments the cassette is pre-filled with one or more of a cell culture, a culture media, a cell wash media, a back flush media, an activation reagent, and/or a vector, including any combination of these. In further embodiments, these various elements can be added later via suitable injection ports, etc. In exemplary embodiments the back flush media suitably contains an anticoagulant, such as ethylenediaminetetraacetic acid (EDTA), to reduce clumping of the target cell population that is transferred from the separation filter.

As described herein, in embodiments, the cassettes suitably further include one or more of a pH sensor 524, a glucose sensor (not shown), an oxygen sensor 526, a carbon dioxide sensor (not shown), a lactic acid sensor/monitor (not shown), and/or an optical density sensor (not shown). See FIG. 5 for exemplary positions within the flowpath. The cassettes can also include one or more sampling ports and/or injection ports. Examples of such sampling ports 220 and injection ports (222) are illustrated in FIG. 2A, and exemplary locations in the flowpath shown in FIG. 5, and can include an access port for connecting the cartridge to an external device, such as an electroporation unit or an additional media source. FIG. 2A also shows the location of the cellular sample input 202, reagent warming bag 224 which can be used to warm cell media, etc., and secondary chamber 230.

In embodiments, cassette 102 suitably includes a low temperature chamber, which can include a refrigeration area 226 suitably for storage of a cell culture media, as well as a high temperature chamber, suitably for carrying out activation, transduction, transfection and/or expansion of a cell culture. Suitably, the high temperature chamber is separated from the low temperature chamber by a thermal barrier. As used herein “low temperature chamber” refers to a chamber, suitably maintained below room temperature, and more suitably from about 4° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette, and connected to the cassette via an access port.

As used herein “high temperature chamber” refers to chamber, suitably maintained above room temperature, and more suitably maintained at a temperature to allow for cell proliferation and growth, i.e., between about 35-40° C., and more suitably about 37° C. In embodiments, high temperature chamber suitably includes cell culture chamber 206 (also called proliferation chamber or cell proliferation chamber throughout).

FIGS. 3A-3B show the COCOON automated cell engineering system 300 with cassette 102 positioned inside (cover of automated cell engineering system opened in FIG. 3B). Also shown is an exemplary user interface, which can include a bar code reader, and the ability to receive using inputs by touch pad or other similar device.

The automated cell engineering systems and cassettes described herein suitably have three relevant volumes, the cell culture chamber volume, the working volume, and the total volume. Suitably, the working volume used in the cassette ranges from 180 mL to 460 mL based on the process step, and can be increased up to about 500 mL, about 600 mL, about 700 mL, about 800 mL, about 900 mL or about 1 L. In embodiments, the cassette can readily achieve 4*10⁹ cells-10*10⁹ cells. The cell concentration during the process varies from 0.3*10⁶ cells/ml to approximately 10*10⁶ cells/ml. The cells are located in the cell culture chamber, but media is continuously recirculated through additional chambers (e.g., crossflow reservoir and satellite volume) to increase the working volume, as described herein.

Fluidics pathways, including gas exchange lines, may be made from a gas-permeable material such as, e.g., silicone. In some embodiments, the automated cell engineering system recirculates oxygen throughout the substantially non-yielding chamber during the cell production methods. Thus, in some embodiments, the oxygen level of a cell culture in the automated cell engineering system is higher than the oxygen level of a cell culture in a flexible, gas-permeable bag. Higher oxygen levels may be important in the cell culture expansion step, as increased oxygen levels may support increased cell growth and proliferation.

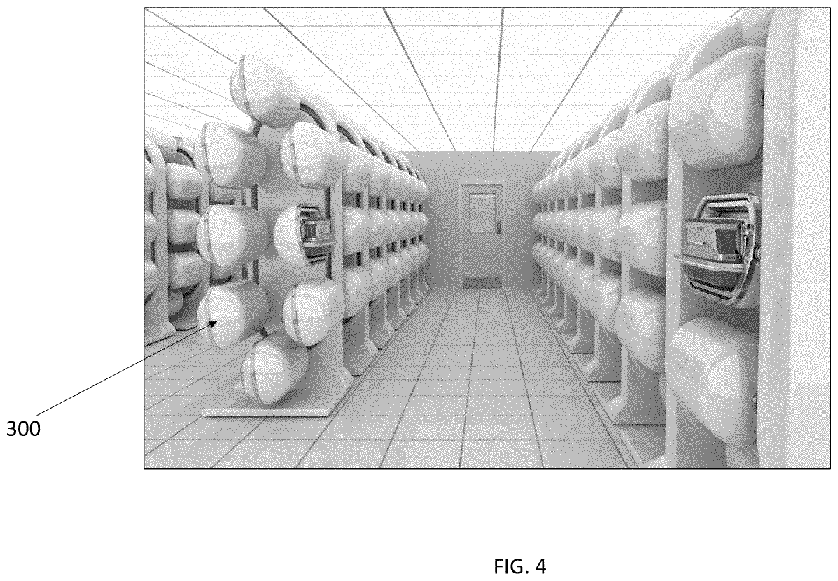

In embodiments, the methods and cartridges described herein are utilized the COCOON platform (Octane Biotech (Kingston, ON)), which integrates multiple unit operations in a single turnkey platform. Multiple cell protocols are provided with very specific cell processing objectives. To provide efficient and effective automation translation, the methods described utilize the concept of application-specific/sponsor-specific disposable cassettes that combine multiple unit operations—all focused on the core requirements of the final cell therapy product. Multiple automated cell engineering systems 300 can be integrated together into a large, multi-unit operation for production of large volumes of cells or multiple different cellular samples for individual patients (see FIG. 4).

In additional embodiments, provided herein is cassette 102 for use in an automated cell engineering system 300. Suitably, the cassette includes cellular sample input 202, cell separation filter 204 fluidly connected to the cellular sample input, the cell separation filter including a matrix which captures immune cells. Cassette 102 further includes cell culture chamber 206 for carrying out activation, transduction, transfection and/or expansion of the immune cells having a chamber volume that is configured to house the immune cells. Cassette 102 also suitably further includes back flush system 514 fluidly connected to the separation filter, and cellular sample output 208 fluidly connected to the cell culture chamber for harvesting the cells. As described herein, suitably the cassette does not include a centrifuge following the cell separation filter (or before the cell separation filter).

In additional embodiments, as described herein, the cassette can further include cell wash system 512 fluidly connected to the separation filter. Suitably, the cassette can further include one or more fluidics pathways connected to the cell culture chamber, wherein the fluidics pathways provide recirculation, removal of waste and homogenous gas exchange and distribution of nutrients to the cell culture chamber suitably without disturbing immune cells within the cell culture chamber. In exemplary embodiments, the fluidic pathways comprise a silicon-based tubing component that allows oxygenation through the tubing component.

In embodiments, the cassette also further includes waste collection chamber 510, suitably following separation filter 204. In additional embodiments, the cassette can include immune cell holding chamber 516, suitably located between the cell separation filter and the cell culture chamber.

As described herein, in embodiments, cell culture chamber 206 is flat and non-flexible chamber, having a low chamber height.

In suitable embodiments, the cassette is pre-filled with culture media, cell wash media, and back flush media, as described herein.

In further embodiments, provided herein is a method of preparing a target cell population for automated processing. As described herein, the methods suitably allow for the introduction of a sample of cells, including a whole blood sample, and then separating out a desired or target cell population from this cell sample for further processing, suitably further automated processing in a automated cell engineering systems, such as those described herein.

In exemplary methods, a cellular sample that contains a target cell population is introduced into cassette 102 of automated cell engineering system 300. As described herein, exemplary cellular samples include blood samples (including whole blood), tissue samples, bodily fluid samples, etc.

In embodiments, as described with reference to FIG. 2A, showing a cassette for carrying out the methods, and FIG. 5 showing a flowpath or flowchart of the cassette process, a cellular sample is suitably introduced at cellular sample input 202. A cellular sample can be introduced for example, from a syringe, container, vial, blood bag, etc.

Following the introduction of the cellular sample, as shown in FIG. 5, in embodiments, the cellular sample passes through control valve (522) V3, and through the fluidic pathways (labeled generically as 540), while being driven by pump 520.

The cellular sample is then suitably passed through separation filter 204, after passing through valve V11. As described herein, cell separation filter 204 suitably includes a matrix for capturing the a desired cell population, including a target cell population from the cellular sample.

In exemplary embodiments, a back flushing occurs, during which cell separation filter 204 is back flushed, suitably from back flush system 512. In such embodiments, a back flush media is contained in back flush system 512, passed through valve V4, and driven via pump 520 through valve V12 and valve V1, to back flush the cell separation filter. This back flushing transfers the target cell population that was captured on the matrix of the cell separation filter, so that the target cell population can be removed from the filter and undergo further processing, including further automated processing. Suitably the back flushing occurs using a back flush media containing an anticoagulant, so as to limit the coagulation of the target cell population as the cells undergo further automated processing procedures.

In embodiments, the target cell population that is removed from the matrix of the cell separation filter can be transferred to a target cell population holding chamber 516, for example by passing through valve V11. In further embodiments, the target cell population that is removed from the matrix of the cell separation filter can be transferred to a transduction system (not shown), a transfection system (i.e., a non-viral method), suitably through a sample port (e.g., R5 or R6), following passing through valves V11 and V9. Exemplary transduction systems are known in the art and exemplary transfection systems include electroporation systems, etc., and can be included within cassette 102 or can be external to cassette 102. In additional embodiments, the target cell population that is removed from the matrix of the cell separation filter can be transferred to cell culture chamber 206, for example, by passing through valve V11 and then valves V5 or V6. As described herein, these various elements following the cell separation filter allow for the target cell population to undergo further automated processing, including transduction, transfection, growth, expansion, etc.

In additional embodiments, the methods can further include washing the captured target cell population on the cell separation filter prior to the back flushing. For example, cell wash system 512, which can be a bag contained within cassette 102 and include a cell wash media, can pass the cell wash media, via pump 520, through valve V4 and V11 to wash the captured target cell population on cell separation filter 204. Suitably, the target cell population remains on the matrix of the cell separation filter, while additional unwanted waste is passed from the cell separation filter into waste collection chamber 510, via valves V1 and V13. In exemplary embodiments, unwanted waste from cellular sample can also pass through the cell separation filter and into waste collection chamber 510 via valves V1 and V13. Suitably additional embodiments allow for the further filtration of a cellular sample by re-passing the waste from the cellular sample back through the cell separation filter, for example by passing through valves V1, V12 and V11, to complete another filtration cycle. Cell washing can also occur via cell wash system 512 by transferring a cell wash media to target cell holding chamber 516, and wash the cells that are being held in the chamber prior to further processing.

In exemplary embodiments, passing the cellular sample through cell separation filter 204 suitable occurs via gravity filtration. That is, no pumping mechanism is used to drive the cellular sample through the cell separation filter. However, in additional embodiments, pump 520 can be used to generate a positive or negative pressure on the cellular sample, so as to drive the sample through the cell separation filter. A syringe or other mechanism can also be used to provide additional positive or negative pressure if desired, to pass the cellular sample through the cell separation filter.

In exemplary embodiments, following the desired automated processing, the target cell population is suitably collected. This collection can occur via sample output 208, or via one of the various sample ports 220.

As described throughout, the cassettes and methods described herein suitably exclude a centrifuge, and the use of centrifugation. Suitably the methods exclude centrifugation following the transferring the target cell population from the cell separation filter, whether that transfer occur directly following capture via the cell separation filter or via a back flush from the cell separation filter. It has been determined that by excluding centrifugation, a target cell population can be separated from a cellular sample via simple filtration, without the need for harsh centrifugation conditions. This includes removing a target cell population from a sample of whole blood.

In further embodiments, however, a magnetic separation process can be utilized to further eliminate and separate undesired cells and debris from a target cell population. In such embodiments, a magnetic bead or other structure, to which a biomolecule (e.g., antibody, antibody fragment, etc.) has been bound, can interact with a target cell. Various magnetic separation methods, including the use of filters, columns, flow tubes or channels with magnetic fields, etc., can then be used to separate the target cell population from undesired cells, debris, etc., that may be in a cellular sample. For example, a target cell population can flow through a tube or other structure and exposed to a magnetic filed, whereby the target cell population is retained or held-up by the magnetic field, allowing undesired cells and debris to pass through the tube. The magnetic field can then be turned off, allowing the target cell population to pass onto a further retention chamber or other area(s) of the cassette for further automated processing.

The flowpath in FIG. 5, also shows the connection between cell culture chamber 206, and a satellite volume 550, which can be provide additional storage capabilities for the cassette, or to increase the overall volume of the automated processes. Also illustrated in FIG. 5 are exemplary positioning of various sensors (e.g., pH sensor 524, dissolved oxygen sensor 526), as well as sampling/sample ports and various valves (including bypass check valves 552), as well as one or more fluidic pathways 540, suitably comprising a silicone-based tubing component, connecting the components. As described herein, use of a silicone-based tubing component allows oxygenation through the tubing component to facilitate gas transfer and optimal oxygenation for the cell culture. Also show in FIG. 5 is the use of one or more hydrophobic filters 554 or hydrophilic filters 556, in the flow path of the cassette.

In additional embodiments, provided herein is an automated cell engineering system 300. As shown in FIGS. 3A and 3B, automated cell engineering system 300 suitably includes an enclosable housing 302, and cassette 102, contained within the enclosable housing. As used herein, “enclosable housing” refers to a structure than can be opened and closed, and within which cassette 102 as described herein, can be placed and integrated with various components such as fluid supply lines, gas supply lines, power, cooling connections, heating connections, etc. As shown in FIGS. 3A and 3B, enclosable housing can be opened (FIG. 3B) to allow insertion of the cassette, and closed (FIG. 3A) to maintain a closed, sealed environment to allow the various automated processes described herein to take place utilizing the cassette.

As described herein, cassette 102 suitably includes cellular sample input 206, cell separation filter 204 fluidly connected to the cellular sample input, cell culture chamber 206 fluidly connected to the cell separation filter, and cellular sample output 208 fluidly connected to the cell culture chamber. As described herein, the cassette (as well as the automated cell engineering system) does not include a centrifuge following the cell separation filter, or suitably in any configuration.

As shown in FIGS. 3A-3B, automated cell engineering system 300 also further includes a user interface 304 for receiving input from a user. User interface 304 can be a touch pad, tablet, keyboard, computer terminal, or other suitable interface, that allows a user to input desired controls and criteria to the automated cell engineering system to control the automated processes and flowpath. Suitably, the user interface is coupled to a computer control system to provide instructions to the automated cell engineering system, and to control the overall activities of the automated cell engineering system. Such instructions can include when to open and close various valves, when to provide media or cell populations, when to increase or decrease a temperature, etc.

As described herein, in embodiments, the cell separation filter includes a matrix which captures a target cell population. Suitably, the matrix captures immune cells.

In embodiments, the cassette in the automated cell engineering systems further comprises a waste collection chamber following the separation filter. A cell wash system fluidly connected to the separation filter, can also be included, as described herein. A back flush system fluidly connected to the separation filter can also be included, as well as optionally a target cell population holding chamber located between the cell separation filter and the cell culture chamber. In embodiments, the cassettes of the automated cell engineering systems further include one or more fluidics pathways, wherein the fluidics pathways provide recirculation, removal of waste and homogenous gas exchange and distribution of nutrients to the cell culture chamber without disturbing cells within the cell culture chamber. In embodiments, the cell culture chamber is flat and non-flexible chamber, having a low chamber height.

In embodiments of the automated cell engineering system, the cassette is pre-filled with culture media, cell wash media, and back flush media (suitably including an anticoagulant). As described herein, in embodiments, the cassette of the automated cell engineering system can further include one or more of a pH sensor, a glucose sensor, an oxygen sensor, a carbon dioxide sensor, and/or an optical density sensor, and in suitable embodiments, one or more sampling ports.

Automation of unit operations in cell therapy production provides the opportunity for universal benefits across allogeneic and autologous cell therapy applications. In the unique scenario of patient-specific, autologous cell products, and even more emphasized by the recent clinical success of these therapies, the advantages of automation are particularly compelling due to the significant micro-lot complexities of small batch GMP compliance, economics, patient traceability and early identification of process deviations. The associated emergence of complex manufacturing protocols draws attention to the fact that the value of end-to-end integration of automated unit operations in micro-lot cell production has not been a point of significant study. However, the expected demand for these therapies following their impending approval indicates that implementation of a fully closed end-to-end system can provide a much needed solution to manufacturing bottlenecks, such as hands-on-time and footprint.

Developers of advanced therapies are encouraged to consider automation early in the rollout of clinical translation and scale up of clinical trial protocols. Early automation can influence protocol development, avoid the need for comparability studies if switching from a manual process to an automated process at a later stage, and provide a greater understanding of the longer-term commercialization route.

In exemplary embodiments, the automated cell engineering systems described herein comprise a plurality of chambers, and wherein each of steps of the various method described herein are performed in a different chamber of the plurality of chambers of the automated cell engineering system, each of the activation reagent, the vector, and cell culture medium are contained in a different chamber of the plurality of the chambers prior to starting the method, and wherein at least one of the plurality of chambers is maintained at a temperature for growing cells (e.g., at about 37° C.) and at least one of the plurality of chambers is maintained at a refrigerated temperature (e.g., at about 4-8° C.).

In embodiments, the automated cell engineering systems described herein are monitored with a temperature sensor, a pH sensor, a glucose sensor, an oxygen sensor, a carbon dioxide sensor, and/or an optical density sensor. Accordingly, in some embodiments, the automated cell engineering system includes one or more of a temperature sensor, a pH sensor, a glucose sensor, an oxygen sensor, a carbon dioxide sensor, and/or an optical density sensor. In additional embodiments, the automated cell engineering system is configured to adjust the temperature, pH, glucose, oxygen level, carbon dioxide level, and/or optical density of the cell culture, based on the pre-defined culture size. For example, if the automated cell engineering system detects that the current oxygen level of the cell culture is too low to achieve the necessary growth for a desired cell culture size, the automated cell engineering system will automatically increase the oxygen level of the cell culture by, e.g., introducing oxygenated cell culture media, by replacing the cell culture media with oxygenated cell culture media, or by flowing the cell culture media through an oxygenation component (i.e., a silicone tubing). In another example, if the automated cell engineering system detects that the current temperature of the cell culture is too high and that the cells are growing too rapidly (e.g., possible overcrowding of the cells may lead to undesirable characteristics), the automated cell engineering system will automatically decrease the temperature of the cell culture to maintain a steady growth rate (or exponential growth rate, as desired) of the cells. In still further embodiments, the automated cell engineering system automatically adjusts the schedule of cell feeding (i.e., providing fresh media and/or nutrients to the cell culture) based on the cell growth rate and/or cell count, or other monitored factors, such as pH, oxygen, glucose, etc. The automated cell engineering system may be configured to store media (and other reagents, such as wash solutions, etc.) in a low-temperature chamber (e.g., 4° C. or −20° C.), and to warm the media in a room temperature chamber or a high-temperature chamber (e.g., 25° C. or 37° C., respectively) before introducing the warmed media to the cell culture.

### Additional Exemplary Embodiments

Embodiment 1 is a cassette for use in an automated cell engineering system, comprising a cellular sample input, a cell separation filter fluidly connected to the cellular sample input, a cell culture chamber fluidly connected to the cell separation filter, and a cellular sample output fluidly connected to the cell culture chamber, wherein the cassette does not include a centrifuge following the cell separation filter.

Embodiment 2 includes the cassette of embodiment 1, wherein the cell separation filter includes a matrix which captures a cell population.

Embodiment 3 includes the cassette of embodiment 1, wherein the matrix captures target cells.

Embodiment 4 includes the cassette of embodiments 1-3, further comprising a waste collection chamber following the cell separation filter.

Embodiment 5 includes the cassette of embodiments 1-4, further comprising a cell wash system fluidly connected to the cell separation filter.

Embodiment 6 includes the cassette of embodiments 1-5, further comprising a back flush system fluidly connected to the cell separation filter, and optionally a target cell population holding chamber located between the cell separation filter and the cell culture chamber.

Embodiment 7 includes the cassette of embodiments 1-6, further comprising one or more fluidics pathways, wherein the fluidics pathways provide recirculation, removal of waste and homogenous gas exchange and distribution of nutrients to the cell culture chamber without disturbing cells within the cell culture chamber.

Embodiment 8 includes the cassette of embodiments 1-7, wherein the cell culture chamber is a flat and non-flexible chamber, having a low chamber height.

Embodiment 9 includes the cassette of embodiments 1-8, wherein the cassette is pre-filled with culture media, cell wash media, and back flush media.

Embodiment 10 includes the cassette of embodiment 9, wherein the back flush media contains an anticoagulant.

Embodiment 11 includes the cassette of embodiments 1-10, further comprising one or more of a pH sensor, a glucose sensor, an oxygen sensor, a carbon dioxide sensor, and/or an optical density sensor.

Embodiment 12 includes the cassette of embodiments 1-11, further comprising one or more sampling ports.

Embodiment 13 is a cassette for use in an automated cell engineering system, comprising a cellular sample input, a cell separation filter fluidly connected to the cellular sample input, the cell separation filter including a matrix which captures immune cells, a cell culture chamber for carrying out activation, transduction and/or expansion of the immune cells having a chamber volume that is configured to house the immune cells, a back flush system fluidly connected to the cell separation filter, and a cellular sample output fluidly connected to the cell culture chamber, wherein the cassette does not include a centrifuge following the cell separation filter.

Embodiment 14 includes the cassette of embodiment 13, further comprising a cell wash system fluidly connected to the cell separation filter.

Embodiment 15 includes the cassette of embodiments 13-14, further comprising one or more fluidics pathways connected to the cell culture chamber, wherein the fluidics pathways provide recirculation, removal of waste and homogenous gas exchange and distribution of nutrients to the cell culture chamber without disturbing immune cells within the cell culture chamber.

Embodiment 16 includes the cassette of embodiments 13-15, further comprising a waste collection chamber following the cell separation filter.

Embodiment 17 includes the cassette of embodiments 13-16, further comprising an immune cell holding chamber located between the cell separation filter and the cell culture chamber.

Embodiment 18 includes the cassette of embodiments 13-17, wherein the cell culture chamber is flat and non-flexible chamber, having a low chamber height.

Embodiment 19 includes the cassette of embodiments 13-18, wherein the cassette is pre-filled with culture media, cell wash media, and back flush media.

Embodiment 20 includes the cassette of embodiments 13-19, wherein one or more of the fluidic pathways comprise a silicon-based tubing component that allows oxygenation through the tubing component.

Embodiment 21 is a method of preparing a target cell population for automated processing, the method comprising introducing a cellular sample containing the target cell population into a cassette of an automated cell engineering system, passing the cellular sample through a cell separation filter, capturing the target cell population from the cellular sample onto a matrix of the cell separation filter, back flushing the cell separation filter; and transferring the target cell population from the cell separation filter, so that the target cell population can undergo automated processing.

Embodiment 22 includes the method of embodiment 21, wherein the transferring comprises transferring the target cell population to a target cell population holding chamber, a transduction system, a system for transfection, or a cell culture chamber, so that the target cell population can undergo automated processing.

Embodiment 23 includes the method of embodiment 22, wherein the transduction system is an electroporation system.

Embodiment 24 includes the method of embodiments 21-23, further comprising washing the captured target cell population on the cell separation filter prior to the back flushing.

Embodiment 25 includes the method of embodiments 21-24, further comprising passing unwanted waste from the cellular sample through the cell separation filter and into a waste collection chamber.

Embodiment 26 includes the method of embodiments 21-25, wherein the passing the cellular sample through the cell separation filter occurs via gravity filtration.

Embodiment 27 includes the method of embodiments 21-26, wherein the method excludes centrifugation following the transferring the target cell population from the cell separation filter.

Embodiment 28 includes the method of embodiments 21-26, further comprising collecting the target cell population from the cassette following the automated processing.

Embodiment 29 is an automated cell engineering system, comprising an enclosable housing, a cassette contained within the enclosable housing, the cassette comprising a cellular sample input, a cell separation filter fluidly connected to the cellular sample input, a cell culture chamber fluidly connected to the cell separation filter, and a cellular sample output fluidly connected to the cell culture chamber, wherein the cassette does not include a centrifuge following the cell separation filter, and a user interface for receiving input from a user.

Embodiment 30 includes the automated cell engineering system of embodiment 29, wherein the cell separation filter of the cassette includes a matrix which captures a cell population.

Embodiment 31 includes the automated cell engineering system of embodiment 30, wherein the matrix captures target cells.

Embodiment 32 includes the automated cell engineering system of embodiments 29-31, wherein the cassette further comprises a waste collection chamber following the cell separation filter.

Embodiment 33 includes the automated cell engineering system of embodiments 29-32, wherein the cassette further comprises a cell wash system fluidly connected to the cell separation filter.

Embodiment 34 includes the automated cell engineering system of embodiments 29-33, wherein the cassette further comprises a back flush system fluidly connected to the cell separation filter, and optionally a target cell population holding chamber located between the cell separation filter and the cell culture chamber.

Embodiment 35 includes the automated cell engineering system of embodiments 29-34, wherein the cassette further comprises one or more fluidics pathways, wherein the fluidics pathways provide recirculation, removal of waste and homogenous gas exchange and distribution of nutrients to the cell culture chamber without disturbing cells within the cell culture chamber.

Embodiment 36 includes the automated cell engineering system of embodiments 29-35, wherein the cell culture chamber of the cassette is flat and non-flexible chamber, having a low chamber height.

Embodiment 37 includes the automated cell engineering system of embodiments 29-35, wherein the cell culture chamber of the cassette is a bag or hard chamber.

Embodiment 38 includes the automated cell engineering system of embodiments 29-37, wherein the cassette is pre-filled with culture media, cell wash media, and back flush media.

Embodiment 39 includes the automated cell engineering system of embodiment 38, wherein the back flush media contains an anticoagulant.

Embodiment 40 includes the automated cell engineering system of embodiments 29-39, wherein the cassette further comprises one or more of a pH sensor, a glucose sensor, an oxygen sensor, a carbon dioxide sensor, and/or an optical density sensor.

Embodiment 41 includes the automated cell engineering system of embodiments 29-40, wherein the cassette further comprises one or more sampling ports.

Embodiment 42 includes the automated cell engineering system of embodiments 29-41, further comprising a computer control system, wherein the user interface is coupled to the computer control system to provide instructions to the automated cell engineering system.

### EXAMPLES

### Example 1—Establishing Cell Filtration for Automated Cell Engineering Systems

The Octane Cocoon™ system is a closed, automated, end-to-end cell engineering system for the manufacture of cell therapy products. The Cocoon™ is comprised of three main components: the base instrument, software, and customizable disposable cassette. The system is capable of automated cell isolation, expansion, concentration, and buffer exchange for both upstream and downstream cell culture processes; however, it does not have centrifugation functionality.

Isolation of target cell populations by adherence can be applied to most adherent cell types including mesenchymal stem cells (MSCs), dendritic cells, and monocytes. For example, human bone marrow MSCs can be isolated by adherence in the Cocoon™ cassette proliferation chamber. 1-2 days post-inoculation of the bone marrow tissue, contaminating red blood cells (RBCs) and other suspension cells are drained to waste, leaving behind adherent cell types in the Cocoon™ cassette proliferation chamber. Media exchanges occurred every 2-3 days with media designed to promote MSC expansion.

Culturing T-cells in the Cocoon™ cassette requires a purified population of either T-cells or peripheral blood mononuclear cells (PBMCs), typically from a whole blood collection from a donor. To eliminate pre-processing procedures required to obtain leukocytes while also decreasing the amount of RBC contamination in the initial Cocoon™ starting material, whole blood filtration was evaluated.

Pall Life Sciences' Arcadis WBC (White Blood Cell) Syringe Filter (Catalog Number AP-4851) and Haemonetics Leukocyte Filter for Salvaged Blood (Catalog Number RS-1) (FIGS. 2B-2C) both contain fibrous matrix and media which captures and retains leukocytes upstream of the filter outlet, while allowing RBCs and other contaminating cells to pass through to waste. The captured leukocytes are then backwashed from the filter and collected for cell culture activities. The Acrodisc WBC Syringe Filters (Pall) can process up to 12 mL of donor whole blood or leukopheresis sample, while the Leukocyte Filter (Haemonetics) can process up to 450 mL of donor whole blood or leukopheresis sample.

By using these filters, or similar, in the fluidic pathway of the Cocoon™ cassette, human T-cells can be isolated from whole blood or Leukopak donor samples for CAR-T and other cell therapy products within the Cocoon™ system. The proposed process flow path described herein allows end users to introduce donor whole blood or leukopheresis samples sterilely and directly into the Cocoon™ system. The whole blood filter can be integrated within the Cocoon™ disposable cassette fluidic pathway to separate leukocytes from the mixed cell population and further expand them in the Cocoon™ proliferation chamber. The final therapeutic product can then be automatically harvested and used fresh or cryopreserved, as required.

### Methods

### Density Gradient Isolation Using Ficoll Plaque Plus (Fisher)

Between 100 mL and 450 mL of whole blood or leukopheresis product was obtained. The initial donor sample was then divided into 2 collections: the first for processing via Ficoll density gradient and the second via a cell separation filter. For density gradient isolation, half of the initial donor sample was processed using standard procedures for the manufacturing of human PBMCs. Specifically, the donor sample was diluted 1:1 in an equal volume of 2 mM EDTA/1×DPBS (Lonza). The diluted sample was then carefully layered in 30 mL fractions onto 15 mL of Ficoll Plaque Plus density gradient solution (GE Healthcare) for a total volume of up to 45 mL per 50 mL conical tube. The tubes were then centrifuge at 400×g for 40 min at room temperature. The top layer of plasma was removed to approximately 10 mL above the buffy coat layer of the tube which contained the PBMCs. The PBMCs were collected and washed in 2 mM EDTA/1×DPBS at three times the collection volume. The collected cells were then counted in duplicate using the Nucleocounter NC-200 (Chemometec), analyzed via flow cytometry (FACS) analysis, and cryopreserved.

Whole Blood Filtration using Acrodisc White Blood Cell Syringe Filter (Pall)

Half of the initial donor whole blood and leukopheresis samples, up to 50 mL, were processed in 6 mL-12 mL fractions using the Acrodisc WBC filters per the manufacturer's instructions. The filter inlet was attached to a 10 mL syringe and mounted over a sterile waste container. 6 mL-12 mL of both diluted and undiluted whole blood and leukopak samples were added to the syringe housing. The samples were then filtered through the WBC filter via gravity. Time to completely filter the samples was recorded. The filter was then washed twice with 5 mL PBS, pH 7.4. To collect the cells, the WBC filter was carefully removed from the syringe housing, a clean 150 mL blood collection bag (WalkMed) attached to the inlet side of the filter, and a media bag filled with 10 mL of PBS (Lonza) was attached to the outlet of the WBC filter. The filter was then back flushed with the PBS and collected in the 150 mL blood collection bag (WalkMed). The collected cell suspension was then washed in 2 mM EDTA/1×DPBS at three times the collection volume. Cells were then counted in duplicate using the Nucleocounter NC-200 (Chemometec) and samples cryopreserved for flow cytometry (FACS) analysis. Cells isolated from the same conditions were then pooled and inoculated in duplicate T-25 tissue flask cultures at 1e⁷ cells in 6 mL of X-VIVO 15 media (Lonza) supplemented with 5% human serum A/B. 100% media exchange and cell counts were performed on all cultures on days 4, 6, 8, and 11.

### Pre-Processing Dilution of Whole Blood Sample

Donor 1: 148 mL of whole blood from a single donor was divided into 2 fractions. One 74 mL fraction was diluted 1:1 in 0.2 mM EDTA/1×DPBS (total 148 mL diluted whole blood) and the second 74 mL fraction was left undiluted. Both undiluted and diluted fractions were then split into two additional fractions at 2×74 mL of diluted whole blood and 2×37 mL of undiluted whole blood to use in both Ficoll separation gradient processing and Pall Acrodisc WBC cell separation filtration processing. The use of undiluted whole blood for Ficoll separation is not a standard laboratory practice and was only included in this evaluation to better understand process limitations. Pall Acrodisc WBC filtration sample volumes were 3 mL undiluted, 6 mL diluted and undiluted, 12 mL diluted and undiluted, and 24 mL diluted.

Donor 2: 279 mL of whole blood from a second donor was divided by leaving 133 mL of whole blood undiluted and diluting the second 145 mL whole blood fraction 1:1 in 0.2 mM EDTA/1×DPBS for a total volume of 290 mL diluted whole blood. 54 mL of the diluted whole blood was processed in triplicate by Ficoll. There were no undiluted samples processed via Ficoll for this donor. The remaining diluted and undiluted whole blood fractions were processed via Pall Acrodisc WBC filtration in 6 mL and 12 mL volumes.

### Pre-Processing of Dilution Leukopak Sample

Donor 1: 127 mL of leukopheresis product from a single donor was divided into 28 mL for unwashed sample filtration and the remaining 99 mL washed by diluting the 99 mL in 400 mL 5 mM EDTA-HBSS, centrifuging, discarding the supernatant, and resuspending the cell pellet in 200 mL of 5 mM EDTA-HBSS. 180 mL of this washed sample was utilized for Ficoll separation density gradient and 20 mL for 6 mL and 12 mL Pall Acrodisc WBC Filtration processing.

### Results

### Processing Time for Leukocyte Separation and Collection

Both undiluted and diluted whole blood fractions for Ficoll density gradient isolation were processed simultaneously. Processing time for 30 mL undiluted whole blood and 74 mL of diluted whole blood samples was approximately 4 hours from time of tube layering to wash of leukocytes/buffy coat. No red blood cell lysis steps were included for any samples.

For Pall WBC cell separation filtration, total processing time for 6 mL-24 mL of whole blood samples ranged between 5 minutes to 20 minutes depending on volume processed and whole blood dilution (Table 1). The fastest processing times were observed when 3 mL of whole blood was diluted 1:1 with 2 mM EDTA/1×DPBS, completely filtering via gravity within 3 minutes and on average an 8 minute±3 minute total processing time (filtration, two washes, and back flush collection). On average, 6 mL of undiluted whole blood required 10 minutes±2 minutes to pass through the filter and 19 minutes±2 minutes total processing time (filtration, two washes, and back flush collection). When diluted 1:1, 6 mL of undiluted whole blood in 6 mL of 2 mM EDTA/1×DPBS (12 mL total volume) required an average of 7 minutes±2 minutes to pass through the filter via gravity and a total processing time of 13 minutes±4 minutes. Only 11 mL of the 12 mL undiluted whole blood passed through the filter for one donor after 18 minutes, and the syringe plunger was required to manually push the remaining volume and wash through the filter. For this donor, a 1:1 dilution of 12 mL of whole blood in 12 mL of 2 mM EDTA/1×PBS (24 mL total volume) clogged after 16 minutes with only ˜11 mL processed. The remaining volume and two subsequent washes were manually pushed through the filter with the plunger of the syringe filter. For the second donor, both 12 mL undiluted whole samples clogged after 30 minutes with 3 mL and 5 mL unprocessed. No manual interventions were attempted for the second donor 12 mL undiluted whole blood volumes nor was a 1:1 dilution performed. One 10 mL undiluted whole blood sample was filtered and completed the gravity filtration of the full volume after 11 minutes and had a total processing time of 19 minutes.

Washed and unwashed leukopak samples clogged the Pall Acrodisc WBC filter after 4 mL of the each 6 mL and 12 mL sample was processed via gravity through the filter (Table 2). Manual intervention was required to process the remaining 2 mL-8 mL of sample using the syringe plunger. Average timing to change process filtration flow for the collection of captured cells was 6-7 minutes.

TABLE 1
Whole Blood Processing Times Summary Table

| | | Time to | | | |
|---|---|---|---|---|---|
| | | Pass | 2 × 5 mL | Time to | Total |
| | | through | PBS Filter | Back | Processing |
| | | filter | Wash | Flush | Time |
| Donor | Sample Condition | (minutes) | (minutes) | (minutes) | (minutes) |
| 1 | Pall Undiluted, 6 mL, 1a | 8 | 9 | | 17 |
| 1 | Pall Undiluted, 6 mL, 1b | 8 | 9 | | 17 |
| 2 | Pall Undiluted, 6 mL, 1a | 9 | 11 | 1 | 21 |
| 2 | Pall Undiluted, 6 mL, 1b | 13 | 6 | 1 | 21 |
| Undiluted, Total Volume 6 mL | | 9.5 | 8.8 | 1.0 | 19.0 |
| Average (min) | | | | | |
| Std. Dev (min) | | 2.4 | 2.1 | 0.0 | 2.3 |
| 1 | Pall Undiluted, 10 mL | 11 | 8 | | 19 |
| 1 | Pall Undiluted, 12 mL | 18 | 2 | | 20 |
| 2 | Pall Undiluted, 12 mL, 2a | 30+ | n/a | n/a | n/a |
| 2 | Pall Undiluted, 12 mL, 2b | 30+ | n/a | n/a | n/a |
| 1 | Pall Diluted, 6 mL, 1b | 3 | 3 | | 6 |
| 2 | Pall Diluted, 6 mL, 1a | 3 | 7 | 1 | 11 |
| 2 | Pall Diluted, 6 mL, 1b | 3 | 7 | 1 | 11 |
| Diluted 1:1, Total Volume 6 mL | | 2.8 | 5.0 | 1.0 | 8.3 |
| Average (min) | | | | | |
| Std. Dev (min) | | 0.5 | 2.3 | 0.0 | 3.2 |
| 1 | Pall Diluted, 12 mL | 6 | 3 | | 9 |
| 2 | Pall Diluted, 12 mL, 2a | 5 | 8 | 1 | 14 |
| 2 | Pall Diluted, 12 mL, 2b | 9 | 7 | 1 | 17 |
| Diluted 1:1, Total Volume 12 mL | | 6.7 | 6.0 | 1.0 | 13.3 |
| Average (min) | | | | | |
| Std. Dev (min) | | 2.1 | 2.6 | 0.0 | 4.0 |
| 1 | Pall Diluted, 24 mL | 16+ | 2 | | 18+ |

TABLE 2
Leukopak Donor 1 Pall Acrodisc WBC Filtration Processing Times

| | | Total |
|---|---|---|
| | Time to Pass | Processing |
| Leukopak Sample | through filter | Time |
| Condition | (minutes) | (minutes) |
| Pall Unwashed, 6 mL | 4 mL processed at 20 minutes. | 95 |
| Pall Unwashed, 12 mL | Backpressure observed. | 98 |
| Pall Washed, 6 mL | Used syringe plunger at 40+ min to | 70 |
| Pall Washed, 12 mL | complete process | 73 |

### Post Processing Cell Yield and Viability

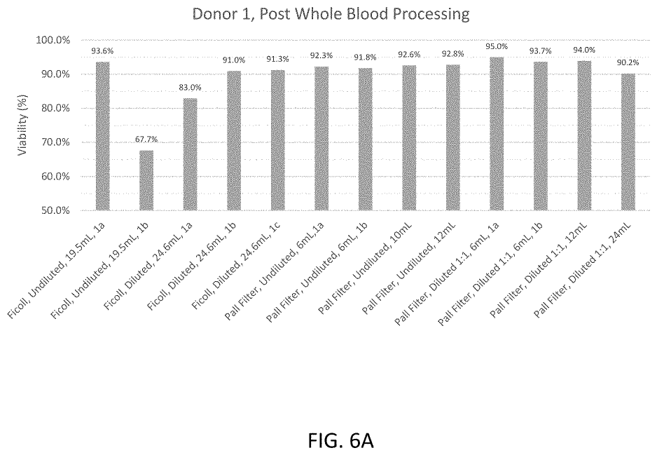

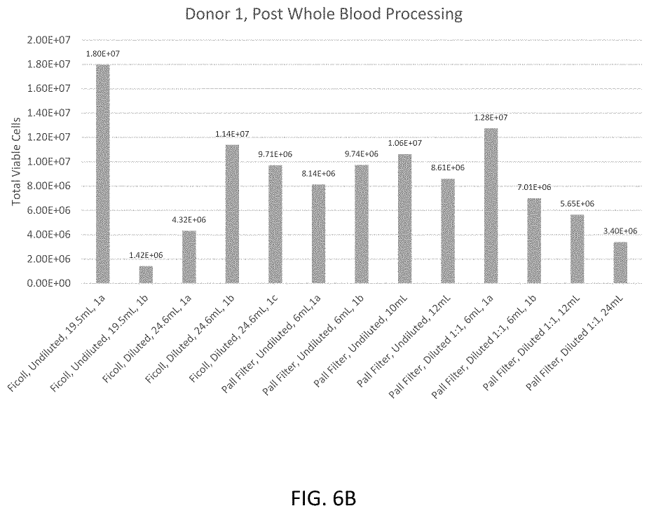

Whole Blood Donor 1: Two Donor 1 whole blood samples were omitted from data analysis shown in Table 3, but are shown in FIGS. 6A and 6B. Of the remaining samples, an average of 1×10⁶ viable cells per mL of processed whole blood was collected via the Pall Acrodisc WBC filter compared to 0.9×10⁶ cells per mL of whole blood processed via Ficoll density gradient separation. 27.3% less viable cells per mL of whole blood was obtained from undiluted Pall Acrodisc WBC filter samples compared to diluted WBC filtered samples. 8% more viable cells per mL of processed whole blood was obtained from undiluted Ficoll samples when compared to diluted Ficoll samples. 15% more viable cells per mL of processed whole blood was obtained from undiluted whole blood when processed via Acrodisc WBC filter compared to undiluted Ficoll processing. 38% more viable cells per mL were obtained when diluted whole blood was processed via Acrodisc WBC filtration compared to Ficoll density gradient methods. Viability were similar for all samples, ranging from 91% to 94%. Even volumes of undiluted blood were carefully layered in the two Ficoll samples. FIGS. 6A and 6B show the difference in cell yield and viability.

TABLE 3
Whole Blood Donor 1 Post Processing Data Summary Table.

| | | Total | Average | |
|---|---|---|---|---|
| | | Cells | cells/mL | Average |
| | | Collected | of initial | Viability |
| Donor 1 | Process | Post | whole blood | Post |
| Sample | Volume | Processing | volume | Processing |
| Condition | (mL) | (cells) | (cells/mL) | (%) |
| Undiluted Pall | 34 | 3.71 × 10⁷ | 1.09 × 10⁶ | 92.4% ± 0.4% |
| Filtration | | | | |
| Diluted Pall | 48 | 3.33 × 10⁷ | 1.39 × 10⁶ | 92.5% ± 1.7% |
| Filtration | | | | |
| Undiluted | 20 | 1.80 × 10⁷ | 0.92 × 10⁶ | 93.6% |
| Ficoll | | | | |
| Diluted Ficoll | 49 | 2.11 × 10⁷ | 0.86 × 10⁶ | 91.1% ± 0.2% |

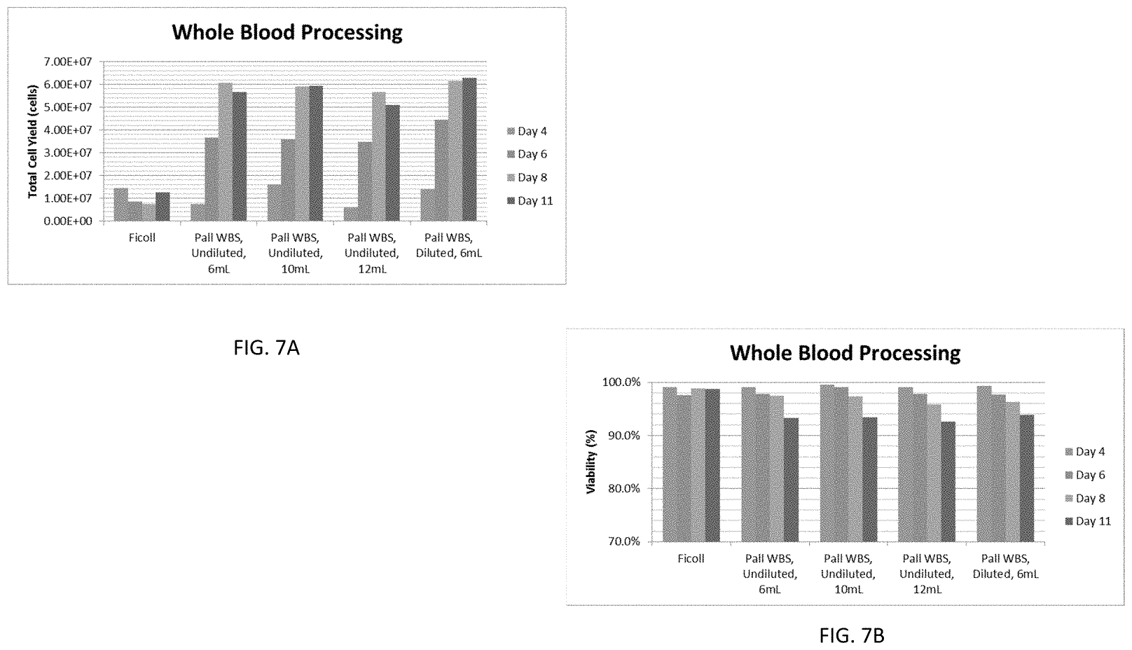

Freshly isolated primary cells from both the filtered and Ficoll processes were inoculated into T-25 tissue culture flasks at a target of 1e⁷ viable cells per flask to mimic expected Cocoon inoculation cell densities. There were not enough cells in the diluted whole blood 12 mL and 24 mL samples to inoculate at the 1e⁷ cell density. By Day 6, all cultures with whole blood filtration isolation procedures were 3-4 fold higher than the initial total cell numbers, with the highest cell numbers achieved from the diluted whole blood, 6 mL filtration sample (FIG. 7A). Cultures inoculated post Ficoll density gradient isolation showed no significant growth from the day of inoculation through harvest, despite maintaining a culture viability at approximately 98% from days 0-11 (FIG. 7B). All cultures maintained viability about 96% in the first 8 days of culture, and above 92% at day 11.

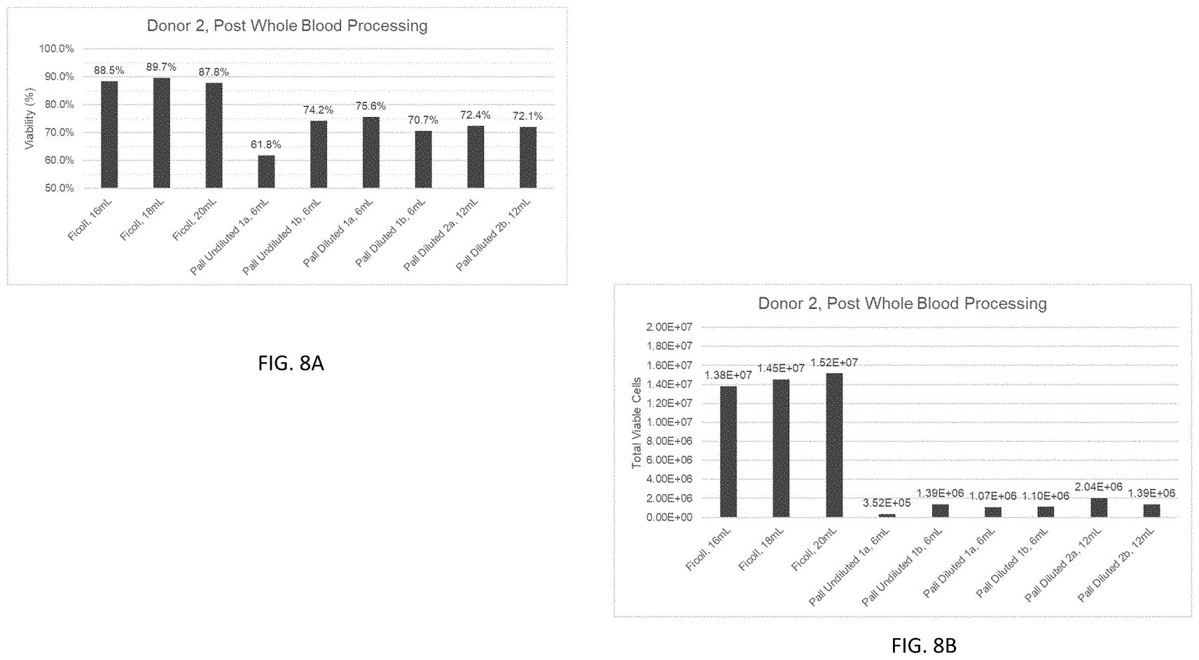

Whole Blood Donor 2: On average, 16.1×10⁵ viable cells per mL of processed whole blood with 88.6%±0.9% viability was obtained via Ficoll separation gradient methods, compared to 3.11×10⁵ with 68.0%±6.2% viability of the undiluted, filtered samples and 3.11×10⁵ with 72.7%±2.1% viability for the diluted, filtered samples (Table 4, FIG. 8A, and FIG. 8B). 53% less viable cells per mL of whole blood was obtained with undiluted Pall Acrodisc WBC filter samples compared to diluted WBC filtered samples, whereas Donor 1 yielded 2% less cells/mL from the undiluted samples compared the diluted samples. 91% less viable cells per mL of processed whole blood was obtained from undiluted whole blood when processed via Acrodisc WBC filter compared to diluted ficoll processing. 81% less viable cells per mL were obtained when diluted whole blood was processed via Acrodisc WBC filtration compared to ficoll density gradient methods.

TABLE 4
Whole Blood Donor 2 Post Processing Data Summary Table.

| | | Total | Average | |
|---|---|---|---|---|
| | | Cells | cells/mL | Average |
| | | Collected | of initial | Viability |
| Donor 2 | Process | Post | whole blood | Post |
| Sample | Volume | Processing | volume | Processing |
| Condition | (mL) | (cells) | (cells/mL) | (%) |
| Undiluted Pall | 12 | 1.74 × 10⁶ | 1.45 × 10⁵ | 68.0% ± 6.2% |
| Filtration | | | | |
| Diluted Pall | 36 | 5.60 × 10⁶ | 3.11 × 10⁵ | 72.7% ± 2.1% |
| Filtration | | | | |
| Diluted Ficoll | 54 | 43.5 × 10⁶ | 16.1 × 10⁵ | 88.6% ± 0.9% |

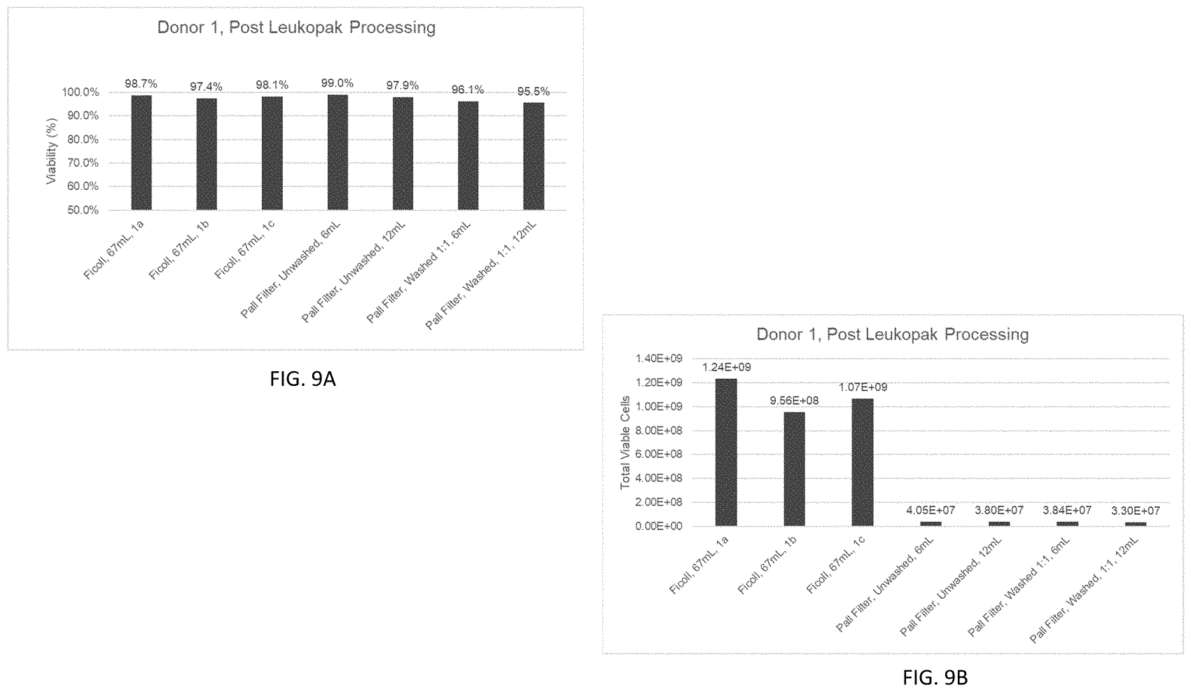

Leukopak Donor 1: On average, 32.9×10⁶ viable cells per mL of Leukopheresis product processed was obtained via Ficoll separation gradient methods at 98.1%±0.6% viability. In comparison, 8.02×10⁶ viable cells per mL of processed washed (1:1 diluted) leukopheresis product and 4.36×10⁶ viable cells of unwashed product was obtained via filtration methods at 95.8%±0.3% viability and 98.4%±0.6% viability, respectively (Table 5, FIG. 9A, and FIG. 9B).

46% less viable cells per mL of leukopheresis product was obtained with unwashed Pall Acrodisc WBC filter samples compared to washed WBC filtered samples. 87% less viable cells per mL of processed whole blood was obtained from unwashed leukopak samples when processed via Acrodisc WBC filter compared to diluted Ficoll processing (FIG. 9B). 76% less viable cells per mL were obtained when washed leukopheresis product was processed via Acrodisc WBC filtration compared to Ficoll density gradient methods.

TABLE 5
Leukopak Donor Post Processing Data Summary Table

| | | Total | Average | |
|---|---|---|---|---|
| | | Cells | cells/mL | Average |
| | | Collected | of initial | Viability |
| Leukopak | Process | Post | whole blood | Post |
| Sample | Volume | Processing | volume | Processing |
| Condition | (mL) | (cells) | (cells/mL) | (%) |
| Unwashed Pall | 18 | 7.85 × 10⁷ | 4.36 × 10⁶ | 98.4% ± 0.6% |
| Filtration | | | | |
| Washed Pall | 18 | 7.14 × 10⁷ | 8.02 × 10⁶ | 95.8% ± 0.3% |
| Filtration | | | | |
| Washed Ficoll | 200 | 326 × 10⁷ | 32.9 × 10⁶ | 98.1% ±0.6% |

FACS analysis of CD3+ T-cell Populations

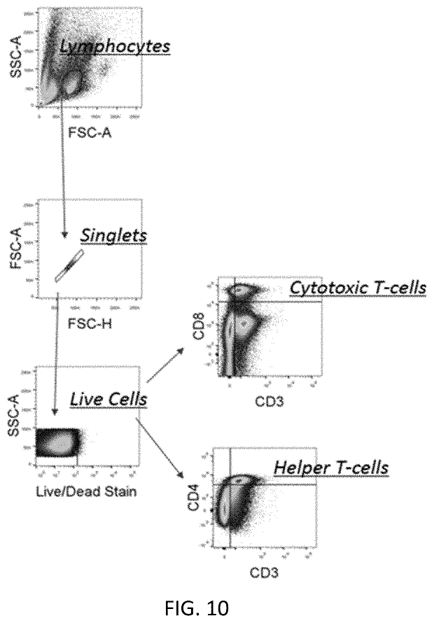

Current Cell Therapies are focused on the optimization of CAR-T cell therapy procedures for automated cell engineering systems, such as the Cocoon™ system. With this in mind, the percentage of CD3+ T-cells, ratio of CD3+CD4+ T-cells to CD3+CD8+ T-cells were compared between whole blood and leukopack cell collection processed by Ficoll and filtration methods (FIG. 10).

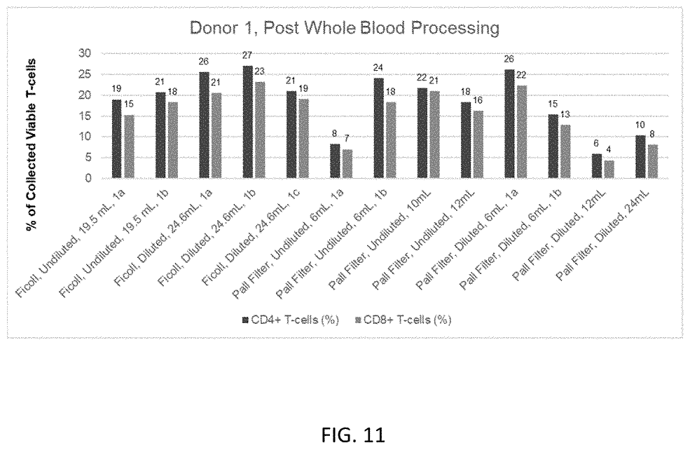

Whole Blood Donor 1: The lowest percentages of CD3+CD4 T-cells (6%-8%) and CD3+CD8+ T-cells (4%-10%) were observed in 1 of 2 undiluted, Pall filtered 6 mL whole blood samples and in both the 12 mL and 24 mL diluted, Pall filtered whole blood samples (FIG. 11). For all other samples, approximately 22%±4% of CD3+CD4+ T-cells and 19%±3% of CD3+CD8+ T-cells were captured in both the Ficoll and Pall filtered whole blood samples. Yet, all samples maintained a ratio of roughly 1:1 CD4+ to CD8+ T-cells captured within each condition.

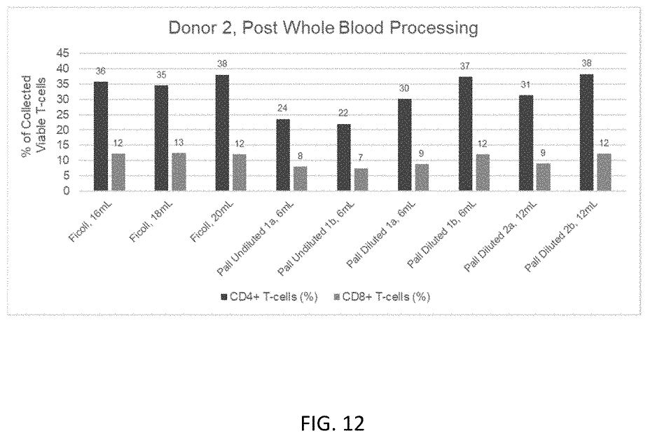

Whole Blood Donor 2: The collected fraction from the undiluted 6 mL whole blood samples presented the lowest percentage of CD3+CD4+ T-cells (23.6% and 22%) and CD3+CD8+ T-cells (8%, and 7.5%) when compared to all other conditions with approximated 30%-38% CD3+CD4+ T-cells and 9%-12% CD3+CD8+ T-cells (FIG. 12). However, all samples showed a 3:1 ratio of CD3+CD4+ cells to CD3+CD8+ cells. Differences in CD4+ to CD8+ ratios between the two whole blood donors is likely a result of donor to donor variability

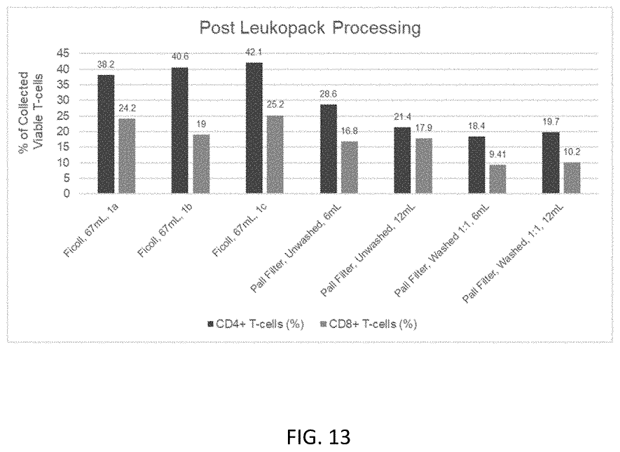

Leukopak Donor Sample: On average, cell fractions from whole blood processed via Ficoll isolation methods contained 40%±2% CD3+CD4+ T-cells and 22.8%±3% CD3+CD8+ T-cells (FIG. 13). This was approximately 15% more CD3+CD4+ T-cells and 5% more CD3+CD8+ T-cells collected than the unwashed filtered Leukopak samples. Ficoll isolation also yielded 21% more CD3+CD4+ T-cells and 13% more CD3+CD8+ T-cells than washed (1:1 diluted) Pall Acrodisc WBC filtered Leukopak samples. With the exception of the 12 mL unwashed filtered Leukopak sample which had a CD3+CD4+ to CD3+CD8+ cell ratio of 1:1, all other ficoll and filtered samples had a 2:1 CD3+CD4+ to CD3+CD8+ ratio. Differences in CD4+, CD8+ yields may have been negatively impacted by the necessity of using the syringe plunger to manually filter the Leukopak samples through the Pall Acrodisc WBC filter, as no more than 4 mL of the Leukopak sample would filter via gravity alone.

### CONCLUSIONS

The methods described herein describe the use of a cell filtration filter, such as a Pall Acrodisc White Blood Cell Syringe Filter, individually or in series, for whole blood leukocyte isolation via the Cocoon system. Updates to implement the use of these filters into the Cocoon™ cassette include:

6 mL-12 mL processing volumes of undiluted or diluted whole blood samples per WBC filter.

Diluting whole blood potentially 1:1 in DPBS or similar buffer for decreased processing times.

Potential for gravity filtration.

The Pall Acrodisc WBC filter makes the capture and expansion of T-cells without centrifugation possible. Larger filters with increased whole blood and leukopheresis product process capabilities are also useful. In particular, the Whole Blood Filtration using Leukocyte Filter for Salvaged Blood by Haemonetics.

### Discussion

Cocoon™ in-line leukocyte isolation from whole blood can be carried out when using specialized filters with leukocyte capture media/matrixes. Suitable Pall or Haemonetics custom filters can be produced for use in the Cocoon™ system.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A cassette for use in an automated cell engineering system, comprising:
(a) a cellular sample input;
(b) a cell separation filter fluidly connected to the cellular sample input;
(c) a cell culture chamber fluidly connected to the cell separation filter; and
(d) a cellular sample output fluidly connected to the cell culture chamber,
wherein the cassette does not include a centrifuge following the cell separation filter.

2. The cassette of claim 1, wherein the cell separation filter includes a matrix which captures a cell population.

3. The cassette of claim 2, wherein the matrix captures target cells.

4. The cassette of claim 1, further comprising a waste collection chamber following the cell separation filter.

5. The cassette of claim 1, further comprising a cell wash system fluidly connected to the cell separation filter.

6. The cassette of claim 1, further comprising a back flush system fluidly connected to the cell separation filter, and optionally a target cell population holding chamber located between the cell separation filter and the cell culture chamber.

7. The cassette of claim 1, further comprising one or more fluidics pathways, wherein the fluidics pathways provide recirculation, removal of waste and homogenous gas exchange and distribution of nutrients to the cell culture chamber without disturbing cells within the cell culture chamber.

8. The cassette of claim 1, wherein the cell culture chamber is a flat and non-flexible chamber, having a low chamber height.

9. The cassette of claim 1, wherein the cassette is pre-filled with culture media, cell wash media, and back flush media.

10. The cassette of claim 9, wherein the back flush media contains an anticoagulant.

11. The cassette of claim 1, further comprising one or more of a pH sensor, a glucose sensor, an oxygen sensor, a carbon dioxide sensor, and/or an optical density sensor.

12. The cassette of claim 1, further comprising one or more sampling ports.

13. A cassette for use in an automated cell engineering system, comprising:
(a) a cellular sample input;
(b) a cell separation filter fluidly connected to the cellular sample input, the cell separation filter including a matrix which captures immune cells;
(c) a cell culture chamber for carrying out activation, transduction and/or expansion of the immune cells having a chamber volume that is configured to house the immune cells;
(d) a back flush system fluidly connected to the cell separation filter; and
(e) a cellular sample output fluidly connected to the cell culture chamber,
wherein the cassette does not include a centrifuge following the cell separation filter.

14. The cassette of claim 13, further comprising a cell wash system fluidly connected to the cell separation filter.

15. The cassette of claim 13, further comprising one or more fluidics pathways connected to the cell culture chamber, wherein the fluidics pathways provide recirculation, removal of waste and homogenous gas exchange and distribution of nutrients to the cell culture chamber without disturbing immune cells within the cell culture chamber.

16. The cassette of claim 13, further comprising a waste collection chamber following the cell separation filter.

17. The cassette of claim 13, further comprising an immune cell holding chamber located between the cell separation filter and the cell culture chamber.

18. The cassette of claim 13, wherein the cell culture chamber is flat and non-flexible chamber, having a low chamber height.

19. The cassette of claim 13, wherein the cassette is pre-filled with culture media, cell wash media, and back flush media.

20. The cassette of claim 15, wherein one or more of the fluidic pathways comprise a silicon-based tubing component that allows oxygenation through the tubing component.

21. A method of preparing a target cell population for automated processing, the method comprising:
(a) introducing a cellular sample containing the target cell population into a cassette of an automated cell engineering system;
(b) passing the cellular sample through a cell separation filter;
(c) capturing the target cell population from the cellular sample onto a matrix of the cell separation filter;
(d) back flushing the cell separation filter; and
(e) transferring the target cell population from the cell separation filter, so that the target cell population can undergo automated processing.

22. The method of claim 21, wherein the transferring comprises transferring the target cell population to a target cell population holding chamber, a transduction system, a system for transfection, or a cell culture chamber, so that the target cell population can undergo automated processing.

23. The method of claim 22, wherein the transduction system is an electroporation system.

24. The method of claim 21, further comprising washing the captured target cell population on the cell separation filter prior to the back flushing.

25. The method of claim 21, further comprising passing unwanted waste from the cellular sample through the cell separation filter and into a waste collection chamber.

26. The method of claim 21, wherein the passing the cellular sample through the cell separation filter occurs via gravity filtration.

27. The method of claim 21, wherein the method excludes centrifugation following the transferring the target cell population from the cell separation filter.

28. The method of claim 21, further comprising collecting the target cell population from the cassette following the automated processing.

29. An automated cell engineering system, comprising:
(a) an enclosable housing;
(b) a cassette contained within the enclosable housing, the cassette comprising:
i. a cellular sample input;
ii. a cell separation filter fluidly connected to the cellular sample input;
iii. a cell culture chamber fluidly connected to the cell separation filter; and
iv. a cellular sample output fluidly connected to the cell culture chamber, wherein the cassette does not include a centrifuge following the cell separation filter; and
(c) a user interface for receiving input from a user.

30. The automated cell engineering system of claim 29, wherein the cell separation filter of the cassette includes a matrix which captures a cell population.

31. The automated cell engineering system of claim 30, wherein the matrix captures target cells.

32. The automated cell engineering system of claim 29, wherein the cassette further comprises a waste collection chamber following the cell separation filter.

33. The automated cell engineering system of claim 29, wherein the cassette further comprises a cell wash system fluidly connected to the cell separation filter.

34. The automated cell engineering system of claim 29, wherein the cassette further comprises a back flush system fluidly connected to the cell separation filter, and optionally a target cell population holding chamber located between the cell separation filter and the cell culture chamber.

35. The automated cell engineering system of claim 29, wherein the cassette further comprises one or more fluidics pathways, wherein the fluidics pathways provide recirculation, removal of waste and homogenous gas exchange and distribution of nutrients to the cell culture chamber without disturbing cells within the cell culture chamber.

36. The automated cell engineering system of claim 29, wherein the cell culture chamber of the cassette is flat and non-flexible chamber, having a low chamber height.

37. The automated cell engineering system of claim 29, wherein the cell culture chamber of the cassette is a bag or hard chamber.

38. The automated cell engineering system of claim 29, wherein the cassette is pre-filled with culture media, cell wash media, and back flush media.

39. The automated cell engineering system of claim 38, wherein the back flush media contains an anticoagulant.

40. The automated cell engineering system of claim 29, wherein the cassette further comprises one or more of a pH sensor, a glucose sensor, an oxygen sensor, a carbon dioxide sensor, and/or an optical density sensor.

41. The automated cell engineering system of claim 29, wherein the cassette further comprises one or more sampling ports.

42. The automated cell engineering system of claim 29, further comprising a computer control system, wherein the user interface is coupled to the computer control system to provide instructions to the automated cell engineering system.
